(19) United States
(12) Patent Application Publication
(10) Pub. No.: US 2002/0107391 A1
(43) Pub. Date: Aug. 8, 2002

(54) Process for the synthesis of an endothelin receptor antagonist

(76) Inventors: Lisa F. Frey, Somerset, NJ (US); Cheng Y. Chen, Plainsboro, NJ (US); Jing Li, Edison, NJ (US); Zhiguo J. Song, Edison, NJ (US); Lushi Tan, Edison, NJ (US); Richard D. Tillyer, Cranford, NJ (US); David M. Tschaen, Holmdel, NJ (US); Matthew M. Zhao, Edison, NJ (US)

Correspondence Address: MERCK AND CO INC, P O BOX 2000, RAHWAY, NJ, 070650907

(21) Appl. No.: 09/982,483

(22) Filed: Oct. 18, 2001

Related U.S. Application Data
(60) Provisional application No. 60/243,171, filed on Oct. 25, 2000.

(51) Int. Cl. C07D 279/08; C07D 265/36; C07D 498/02

(52) U.S. Cl. 544/47; 544/105; 544/253; 544/235; 544/349; 546/112; 548/152; 548/217; 548/302.7; 548/360.1; 548/452

(57) ABSTRACT

The present invention relates to a practical and efficient way to synthesize the compound for the endothelin receptor antagonist involving a Grignard addition and a cyclization reaction to give a desired compound of the general formula shown below:

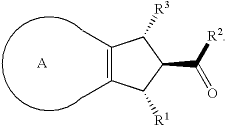

### FIELD OF THE INVENTION

The present invention is directed to a process for preparing an endothelin receptor antagonist in a practical and efficient way.

### BACKGROUND OF THE INVENTION

The endothelin antagonist compound possessing a high affinity for at least one of two receptor subtypes are responsible for the dilation of smooth muscle, such as blood vessels or in the trachea. The endothelin antagonist compounds provide a potentially new therapeutic target, particularly for the treatment of hypertension, pulmonary hypertension, Raynaud's disease, acute renal failure, myocardial infarction, angina pectoris, cerebral infarction, cerebral vasospasm, arteriosclerosis, asthma, gastric ulcer, diabetes, restenosis, prostatauxe endotoxin shock, endotoxin-induced multiple organ failure or disseminated intravascular coagulation, and/or cyclosporin-induced renal failure or hypertension.

Endothelin is a polypeptide composed of amino acids, and it is produced by vascular endothelial cells of human or pig. Endothelin has a potent vasoconstrictor effect and a sustained and potent pressor action (Nature, 332, 411-415 (1988)).

Three endothelin isopeptides (endothelin-1, endothelin-2 and endothelin-3), which resemble one another in structure, exist in the bodies of animals including human, and these peptides have vasoconstriction and pressor effects (Proc. Natl. Acad. Sci., USA, 86, 2863-2867 (1989)).

As reported, the endothelin levels are clearly elevated in the blood of patients with essential hypertension, acute myocardial infarction, pulmonary hypertension, Raynaud's disease, diabetes or atherosclerosis, or in the washing fluids of the respiratory tract or the blood of patients with asthmaticus as compared with normal levels (Japan J. Hypertension, 12, 79, (1989); J. Vascular medicine Biology, 2, 207 (1990); Diabetologia, 33, 306-310 (1990); J. Am. Med. Association, 264, 2868 (1990); and The Lancet, ii, 747-748 (1989) and ii, 1144-1147 (1990)).

Further, an increased sensitivity of the cerebral blood vessel to endothelin in an experimental model of cerebral vasospasm (Japan. Soc. Cereb. Blood Flow & Metabol., 1, 73 (1989)), an improved renal function by the endothelin antibody in an acute renal failure model (J. Clin. Invest., 83, 1762-1767 (1989), and inhibition of gastric ulcer development with an endothelin antibody in a gastric ulcer model (Extract of Japanese Society of Experimental Gastric Ulcer, 50 (1991)) have been reported. Therefore, endothelin is assumed to be one of the mediators causing acute renal failure or cerebral vasospasm following subarachnoid hemorrhage.

Further, endothelin is secreted not only by endothelial cells but also by tracheal epithelial cells or by kidney cells (FEBS Letters, 255, 129-132 (1989); and FEBS Letters, 249, 42-46 (1989)).

Endothelin was also found to control the release of physiologically active endogenous substances such as renin, atrial natriuretic peptide, endothelium-derived relaxing factor (EDRF), thromboxane A₂, prostacyclin, noradrenaline, angiotensin II and substance P (Biochem. Biophys. Res. Commun., 157, 1164-1168 (1988); Biochem. Biophys. Res. Commun., 155, 20 167-172 (1989); Proc. Natl. Acad. Sci. USA, 85 1 9797-9800 (1989); J. Cardiovasc. Pharmacol., 13, S89-S92 (1989); Japan J. Hypertension, 12, 76 (1989); and Neuroscience Letters, 102, 179-184 (1989)). Further, endothelin causes contraction of the smooth muscle of gastrointestinal tract and the uterine smooth muscle (FEBS Letters, 247, 337-340 (1989); Eur. J. Pharnacol., 154, 227-228 (1988); and Biochem. Biophys. Res. Commun., 159, 317-323 (1989)). Further, endothelin was found to promote Go proliferation of rat vascular smooth muscle cells, suggesting a possible relevance to the arterial hypertrophy (Atherosclerosis, 78, 225-228 (1989)). Furthermore, since the endothelin receptors are present in a high density not only in the peripheral tissues but also in the central nervous system, and the cerebral administration of endothelin induces a behavioral change in animals, endothelin is likely to play an important role for controlling nervous functions (Neuroscience Letters, 97, 276-279 (1989)). Particularly, endothelin is suggested to be one of mediators for pain (Life Sciences, 49, PL61-PL65 (1991)).

Internal hyperplastic response was induced by rat carotid artery balloon endothelial denudation. Endothelin causes a significant worsening of the internal hyperplasia (J. Cardiovasc. Pharnacol., 22, 355-359 & 371-373(1993)). These data support a role of endothelin in the pathogenesis of vascular restenosis. Recently, it has been reported that both ET_{A} and ET_{B} receptors exist in the human prostate and endothelin produces a potent contraction of it. These results suggest the possibility that endothelin is involved in the pathophysiology of benign prostatic hyperplasia (J. Urology, 151, 763-766(1994); Molecular Pharmocol., 45, 306-311 (1994)).

On the other hand, endotoxin is one of potential candidates to promote the release of endothelin. Remarkable elevation of the endothelin levels in the blood or in the culture supernatant of endothelial cells was observed when endotoxin was exogenously administered to animals or added to the culture endothelial cells, respectively. These findings suggest that endothelin is an important mediator for endotoxin-induced diseases (Biochem. Biophys. Commun., 161, 1220-1227 (1989); and Acta Physiol. Scand., 137, 317-318 (1989)).

Further, it was reported that cyclosporin remarkably increased endothelin secretion in the renal cell culture (LLC-PKL cells) (Eur. J. Pharmacol., 180, 191-192 (1990)). Further, dosing of cyclosporin to rats reduced the glomerular filtration rate and increased the blood pressure in association with a remarkable increase in the circulating endothelin level. This cyclosporin-inducea renal failure can be suppressed by the administration of endothelin antibody (Kidney Int., 37, 1487-1491 (1990)). Thus, it is assumed that endothelin is significantly involved in the pathogenesis of the cyclosporin-induced diseases. Such various effects of endothelin are caused by the binding of endothelin to endothelin receptors widely distributed in many tissues (Am. J. Physiol., 256, R856-R866 (1989)).

It is known that vasoconstriction by the endothelin is caused via at least two subtypes of endothelin receptors (J. Cardiovasc. Pharmacol., 17 (Suppl.7), S 119-SI21(1991)). One of the endothelin receptors is ET_{A} receptor selective to ET-1 rather than ET-3, and the other is ET_{B} receptor equally active to ET-1 and ET-3. These receptor proteins are reported to be different from each other (Nature, 348, 730-735 (1990)).

These two subtypes of endothelin receptors are differently distributed in tissues. It is known that the ET_{A} receptor is present mainly in cardiovascular tissues, whereas the ET_{B} receptor is widely distributed in various tissues such as brain, kidney, lung, heart and vascular tissues.

Substances that specifically inhibit the binding of endothelin to the endothelin receptors are believed to antagonize various pharmacological activities of endothelin and to be useful as a drug in a wide field. Since the action of the endothelin is caused via not only the ET_{A} receptor but also the ET_{B} receptor, novel non-peptidic substances with ET receptor antagonistic activity to either receptor subtype are desired to block activities of the endothelin effectively in various diseases.

Endothelin is an endogenous substance which directly or indirectly (by controlling liberation of various endogenous substances) induces sustained contraction or relaxation of vascular or non-vascular smooth muscles, and its excess production or excess secretion is believed to be one of pathogeneses for hypertension, pulmonary hypertension, Raynaud's disease, bronchial asthma, gastric ulcer, diabetes, arteriosclerosis, restenosis, acute renal failure, myocardial infarction, angina pectoris, cerebral vasospasm and cerebral infarction. Further, it is suggested that endothelin serves as an important mediator involved in diseases such as restenosis, prostatauxe, endotoxin shock, endotoxin-induced multiple organ failure or disseminated intravascular coagulation, and cyclosporin-induced renal failure or hypertension. Two endothelin receptors ET_{A} and ET_{B} are known so far and antagonists of these receptors have been shown to be potential drug targets.

EP 0526708 A1 and WO 93/08799 A1 are representative examples of patent applications disclosing non-peptidic compounds with alleged activity as endothelin receptor antagonists.

Tillyer et al. (U.S. Pat. No. 5,998,625) is directed to a process for preparing a key intermediate in the synthesis of an endothelin antagonist using a chiral additive to effect an asymmetric conjugate addition.

Tillyer et al. (U.S. Pat. No. 6,046,327) discloses the phosphate-mediated cyclization process in the preparation of an endothelin antagonist.

Ishikawa et al. (WO9505374) discloses fused heteroaromatic cyclopentene derivative having endothelin-antagonist activity.

Bradsher et al. (J. Org. Chem., 46, 1384-1388 (1981), “Oxygen Heterocycles by the Parham Cyclialkylation”) relates to the Parham cyclialkylation to form rings containing oxygen atom to afford 2,3-dihydrobenzofurans, 3,4-dihydro-2H— 1-benzopyrans, or 2,3,4,5-tetrahydro-1-benzoxepins.

An object of the present invention is to develop a practical synthetic route to prepare an asymmetric endothelin receptor antagonist.

### SUMMARY OF THE INVENTION

The present invention relates to a process for preparing a compound for an endothelin receptor antagonist of Formula I,

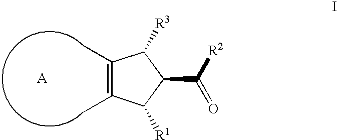

wherein:

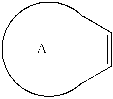

represents:

(a) 5- or 6-membered heterocyclyl containing one to three double bonds, but at least one double bond and 1 to 3 heteroatoms selected from O, N and S, and the heterocyclyl is optionally substituted with one to three substituents 4 selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;

(b) 5- or 6-membered carbocyclyl containing one or two double bonds, but at least one double bond, and the carbocyclyl is optionally substituted with one 4 to three substituents selected from the group consisting of: OH, CO₂R, Br, Cl, F, I, CF₃, N(R⁵)₂, (C₁-C₈)-alkoxy, (C₁-C₈)-alkyl, (C₂-C₈)-alkenyl, (C₂-C₉) alkynyl, (C₃-C₈)-cycloalkyl, CO(CH₂)ₙCH₃, and CO(CH₂)ₙCH₂N(R⁵)₂; or

(c) aryl, wherein aryl is defined as phenyl or naphthyl, which is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂, or when aryl is substituted on adjacent carbons they can form a 5- or 6-membered fused ring having one to three heteroatoms selected from O, N, and S, this ring being optionally substituted on carbon or nitrogen with one to three substituents selected from the group consisting of: H, OH, CO₂R⁶, Br, Cl, F, I, CF₃, N(R⁷)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂), CH₃, and CO(CH₂)ₙCH₂N(R⁵)₂;

and wherein (C₁-C₈)-alkoxy, (C₁-C₈)-alkyl, (C₂-C₈)-alkenyl, (C₂-C₈)-alkynyl, or (C₃-C₈)-cycloalkyl substituent of aryl is further optionally substituted with one to three substituents

selected from the group consisting of: OH, CO₂R⁴, Br, Cl, F, I, CF₃, OCPh₃, N(R⁵)₂, (C₁-C₈)-alkoxy, (C₃-C₈)-cycloalkyl, CO(CH₂)ₙCH₃, and CO(CH₂)ₙCH₂N(R⁵)₂;

R¹ is:

(a) (C₁-C₈)-alkyl, (C₂-C₈)-alkenyl, (C₂-C₈)-alkynyl, or (C₃-C₈)-cycloalkyl,

(b) aryl, wherein aryl as defined above, or

(c) heteroaryl, wherein heteroaryl is defined as a 5- or 6-membered aromatic ring containing one to three heteroatoms selected from O, N and S, and is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;

R² is: OR⁴ or N(R⁵)₂;

R³ is:

(a) (C₁-C₈)-alkyl,

(b) (C₂-C₈)-alkenyl,

(c) (C₂-C₈)-alkynyl,

(d) (C₃-C₇)-cycloalkyl,

(e) aryl, wherein aryl as defined above,

(f) heteroaryl, wherein heteroaryl as defined above

(g) —CHO,

(h) —CO—(C₁-C₈)-alkyl,

(i) —CO-aryl,

(j) —CO-heteroaryl, or

(k) —CO₂R⁴;

n is: 0 to 5;

t is: 0, 1 or 2;

R⁴ is: H, or (C₁-C₈)-alkyl;

R⁵ is: H, (C₁-C₈)-alkyl or aryl, wherein aryl as defined above;

R⁶ is: H, (C₁-C₈)-alkyl or aryl, wherein aryl as defined above; and

R⁷ is: H, (C₁-C₈)-alkyl, aryl or alkyl, wherein aryl is optionally substituted with one to three substituents selected from the group consisting of: OH, CO₂R⁴, Br, Cl, F, I, CF₃, N(R⁵)₂(C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂, or when two R substituents are on the same nitrogen they can join to form a ring of 3 to 6 atoms;

comprising the steps of:

(1) reacting a Grignard reagent with a conjugate adduct compound of Formula II,

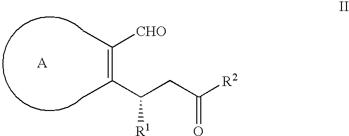

in the presence of a first aprotic solvent and optionally an additive at a temperature range of about −80° C. to about 30° C. to give a Grignard addition product of Formula III; and

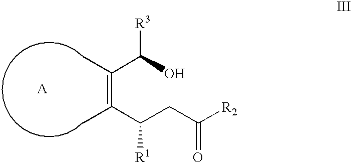

(2) adding phosphoramide reagent to a mixture of the Grignard addition product of Formula III, a second aprotic solvent and a base at a temperature range of about −80° C. to about 30° C. to produce the desired compound of Formula I.

### DETAILED DESCRIPTION OF THE INVENTION

The present invention relates to a novel way to synthesize the compound for the endothelin receptor antagonist involving a Grignard addition and a cyclization to give a desired compound of endothelin receptor antagonist.

The present invention discloses a process for preparing a compound of Formula I,

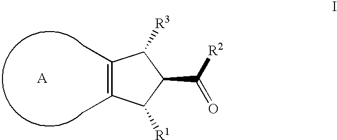

wherein:

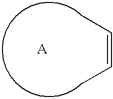

represents:

(a) 5- or 6-membered heterocyclyl containing one to three double bonds, but at least one double bond and 1 to 3 heteroatoms selected from O, N and S, and the heterocyclyl is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;

(b) 5- or 6-membered carbocyclyl containing one or two double bonds, but at least one double bond, and the carbocyclyl is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂; or

(c) aryl, wherein aryl is defined as phenyl or naphthyl, which is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂) CH₂N(R⁵)₂, or when aryl is substituted on adjacent carbons they can form a 5- or 6-membered fused ring having one to three heteroatoms selected from O, N, and S, this ring being optionally substituted on carbon or nitrogen with one to three substituents selected from the group consisting of: H, OH, CO₂R⁶, Br, Cl, F, I, CF₃, N(R⁷)₂, (C₁-C₈)-alkoxy, (C,-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;

and wherein (C₁-C₈)-alkoxy, (C₁-C₈)-alkyl, (C₂-C₈)-alkenyl, (C₂-C₈)-alkynyl, or (C₃-C₈)-cycloalkyl substituent of aryl is further optionally substituted with one to three substituents

selected from the group consisting of: OH, CO₂R⁴, Br, Cl, F, I, CF₃, OCPh₃, N(R⁵)₂, (C₁-C₈)-alkoxy, (C₃-C₈)-cycloalkyl, CO(CH₂)ₙCH₃, and CO(CH₂)ₙCH₂N(R⁵)₂;

R¹ is:

(a) (C₁-C₈)-alkyl, (C₂-C₈)-alkenyl, (C₂-C₈)-alkynyl, or (C₃-C₈)-cycloalkyl,

(b) aryl, wherein aryl as defined above, or

(c) heteroaryl, wherein heteroaryl is defined as a 5- or 6-membered aromatic ring containing one to three heteroatoms selected from O, N and S, and is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;

R² is: OR⁴ or N(R⁵)₂;

R³ is:

(a) (C₁-C₈)-alkyl,

(b) (C₂-C₈)-alkenyl,

(c) (C₂-C₈)-alkynyl,

(d) (C₃-C₇)-cycloalkyl,

(e) aryl, wherein aryl as defined above,

(f) heteroaryl, wherein heteroaryl as defined above,

(g) —CHO,

(h) —CO—(C₁-C₈)-alkyl,

(i) —CO-aryl,

(j) —CO-heteroaryl, or

(k) —CO₂R⁴;

n is: 0 to 5;

t is: 0, 1 or 2;

R⁴ is: H, or (C₁-C₈)-alkyl;

R⁵ is: H, (C₁-C₈)-alkyl or aryl, wherein aryl as defined above;

R⁶ is: H, (C₁-C₈)-alkyl or aryl, wherein aryl as defined above; and

R⁷ is: H,(C—C₈)-alkyl, aryl or alkyl, wherein aryl is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂, or when two R⁷ substituents are on the same nitrogen they can join to form a ring of 3 to 6 atoms;

comprising the steps of:

(1) reacting a Grignard reagent with a conjugate adduct compound of Formula II,

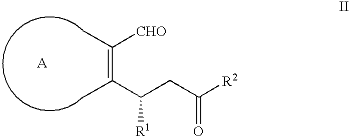

in the presence of a first aprotic solvent and optionally an additive at a temperature range of about −80° C. to about 30° C. to give a Grignard addition product of Formula III; and

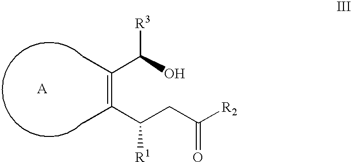

(2) adding phosphoramide reagent to a mixture of the Grignard addition product of Formula III, a second aprotic solvent and a base at a temperature range of about −80° C. to about 30° C. to produce the desired compound of Formula I.

A preferred embodiment of the present invention is a process for preparing a compound of Formula Ia,

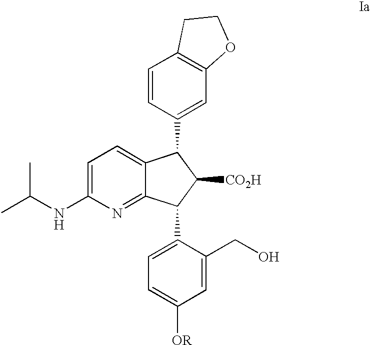

wherein R is independently H or (C₁-C₆)-alkyl comprising the steps of:

(1) reacting ArMgX reagent with a conjugate adduct of Formula IIa,

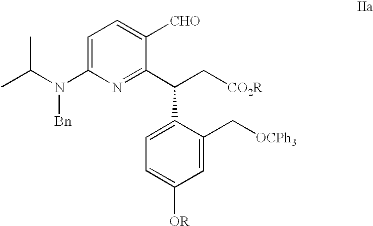

in the presence of a first aprotic solvent at a temperature range of about −80° C. to about 30° C. to give a Grignard addition product of Formula IIa, and

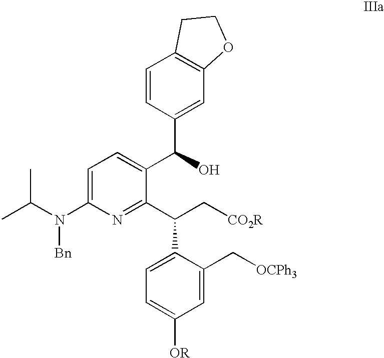

(2) adding phosphoramide reagent to a mixture of the Grignard addition product of Formula IIa in a second aprotic solvent and a base at a temperature range of about −80° C. to about 30° C. to produce the desired compound of Formula Ia.

Another preferred embodiment of the present invention is a process for preparing a compound of Formula Ia,

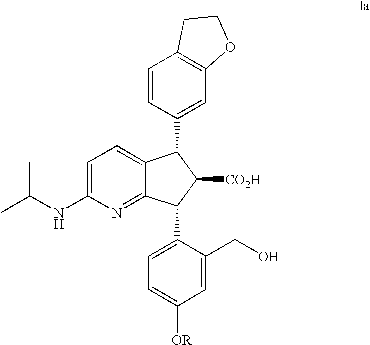

wherein R is independently H or (C₁-C₆)-alkyl comprising the steps of:

(1) reacting an α, β-unsaturated ester

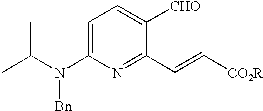

with a chiral auxiliary (S,S)-pseudoephedrine followed by treatment with an aryllithium compound

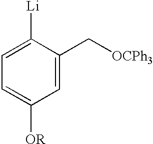

in toluene or tetrahydrofuran or a mixture thereof at a temperature range of about −80° C. to about 0° C. to give a conjugate adduct of Formula IIa,

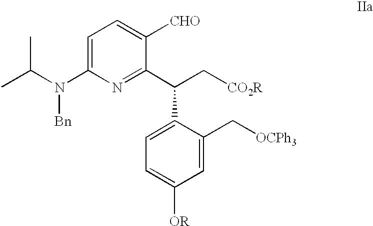

(2) reacting the conjugate adduct of Formula IIa with

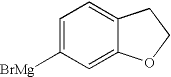

at a temperature range of about −80° C. to about 30° C. to give a Grignard addition product of Formula IIIa,

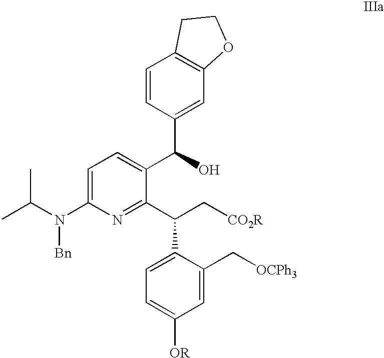

(3) adding phosphoramide reagent to a mixture of the Grignard addition product of Formula E[a in the presence of tetrahydorfuran or a mixture of tetrahydrofuran and toluene, and a base at a temperature range of about −80° C. to about 30° C. to produce a cyclized compound of Formula IV, and

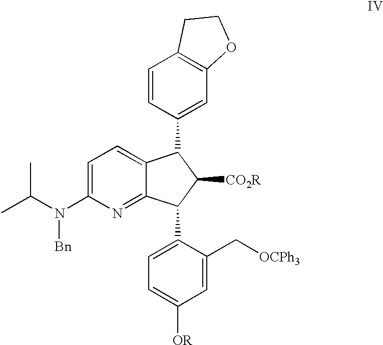

(4) removing protecting groups on the cyclized compound of Formula IV to give the desired compound of Formula Ia.

The process as recited above, wherein the first or second aprotic solvent is selected from the group consisting of tetrahydrofuran, acetonitrile, dimethylacetamide, dimethylformamide, diethyl ether, N-methylpyrrolidinone, dichloromethane, methyl t-butyl ether, toluene, benzene, hexane, pentane, dioxane, and a mixture thereof. A preferred first aprotic solvent is a 1:1 mixture of N-methylpyrrolidinone and tetrahydrofuran at temperature range of about −40° C. to about −50° C. or N-methylpyrrolidinone at temperature range of about −20° C. to about −10° C. A preferred second aprotic solvent is THF or a mixture of TBF/toluene.

The process as recited above, wherein the additive is selected from the group consisting of MgBr₂.Et₂O, LiBr, BF₃.ET₂O, ArLi, and DMPU.

The process as recited above, wherein the Grignard reagent is ArMgX, which is prepared from ArX and Mg.

The process as recited above, wherein the Grignard reagent is

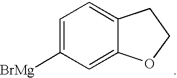

The process as recited above, wherein ArX is prepared by the following steps:

(a) reacting

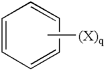

with HO(CH₂)ₘOH in the presence of a O(CH₂)ₘOH

base to give

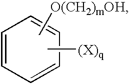

wherein q is 1 to 5, m is 2, 3, or 4 and X is Br, Cl, F, or I;

(b) halogenating —O(CH₂)ₘOH substituent of the benzene to produce the benzene with O(CH₂)ₘX substituent in the presence of an aprotic solvent, water, and halogenating agent at a temperature range of about 0° C. to about 90° C.; and

(c) cyclizing the compound produced in step (b) in the presence of alkyl lithium or aryl lithium to give ArX.

The process as recited above, wherein the ArX is 6-bromo-2,3-dihydrobenzofuran.

The process as recited above, wherein the temperature range in Grignard addition reaction is about −40° C. to about −50° C.

The process as recited above, wherein the phosphoramide reagent is N,N,N,N-tetra(C₁-C₆)-alkylphosphorodiamidic halide or N,N,N,N-tetraarylphosphorodiamidic halide, preferably N,N,N,N-tetramethylphosphorodiamidic chloride, [(CH₃)₂N]₂POCl or N,N,N,N-tetramethylphosphorodiamidic bromide, [(CH₃)₂N]₂POBr, N,N,N,N-tetraethylphosphorodiamidic chloride, [(CH₃CH₂)₂N]₂POCl or N,N,N,N-tetraethylphosphorodiamidic bromide, [(CH₃CH₂)₂N]₂POBr N,N,N,N-tetraisopropylphosphorodiamidic chloride [((CH₃)₂CH)₂N]₂POCl or N,N,N,N-tetraisopropylphosphorodiamidic bromide, [((CH₃)₂CH)₂N]₂POBr, N,N,N,N-tetraphenylphosphorodiamidic chloride, or N,N,N,N-tetraphenylphosphorodiamidic bromide.

The process as recited above wherein the base is selected from the group consisting of n-butyl lithium, phenyl lithium, potassium tert-butoxide, sodium hydride, lithium dilsopropylamide, lithium diethylamide, lithium dimethylamide, potassium hexamethyldisilazide, sodium hexamethyldisilazide, and lithium hexamethyldisilazide. The preferred base is sodium hexamethyldisilazide, which is present in amounts between about 1 equivalent and about 6 equivalents relative to the amount of the phosphoramide reagent or N,N,N′,N′-tetramethylphosphorodiamidic chloride.

The process as recited above, wherein the temperature range for the cyclization in the presence of phosphoramide reagent is about −20° C. to about 25° C.

The process as recited above, which further comprises the steps of:

(a) deprotecting the cyclized compound of Formula IV by removing protecting groups with acid at a temperature range of about 0° C. to about 25° C.;

(b) crystallizing the deprotected compound as benzylamine salt; and

(c) hydrogenating the deprotected compound in the presence of a hydrogenation catalyst and a protic solvent at a temperature range of about 25° C. to about 40° C.

The process as recited above, wherein the hydrogenation catalyst is Pd/C.

The process as recited above, wherein the protic solvent is selected from the group consisting of (C₁-C₆)-alcohol, H₂O, and a mixture thereof. The preferred protic solvent is methanol.

It is further understood that the substituents recited above would include the definitions recited below.

As used herein, the term “alkyl,” unless otherwise indicated, includes those alkyl groups of a designated number of carbon atoms of either a straight, branched, or cyclic configuration. Examples of “alkyl ” include methyl, ethyl, propyl, isopropyl, butyl, sec-butyl, tert-butyl, pentyl, hexyl, heptyl, neopentyl, isopentyl, and the like.

Cycloalkyl denotes rings composed of 3 to 8 methylene groups, each of which may be optionally substituted with other hydrocarbon substituents. Examples of cycloalkyls include, but are not limited to: cyclopropyl, cyclobutyl, cyclopentyl, cyclohexyl, 4-methylcyclohexyl, cycloheptyl, and the like.

The term “alkenyl” includes hydrocarbon chains of a specified number of carbon atoms of either a straight or branched configuration and at least one unsaturation, which may occur at any point along the chain, such as ethenyl, propenyl, butenyl, pentenyl, vinyl, allyl, 2-butenyl and the like.

The term “alkoxy” represents an alkyl group of indicated number of carbon atoms attached through an oxygen bridge, such as methoxy, ethoxy, propoxy, isopropoxy, butoxy, tert-butoxy, pentoxy, and the like.

The term “aryl,” unless specifically defined otherwise, is defined as phenyl and 1-naphthyl or 2-naphthyl, including aryl substituted with a 5- or 6-membered fused ring, such as an unsubstituted and substituted 2,3-dihydrobenzofuran, methylenedioxy, oxazolyl, imidazolyl, or thiazolyl ring. Aryl as defined above may be optionally substituted with one to three of the substituents as set forth in the embodiments recited above.

The heteroaryl substituents represent but are not limited to: a carbazolyl, furanyl, thienyl, pyrrolyl, isothiazolyl, imidazolyl, isoxazolyl, thiazolyl, oxazolyl, pyrazolyl, pyrazinyl, pyridyl, pyrimidyl, and purinyl.

The heterocyclyl substituents represent but are not limited to: oxazolidinyl, thiazolidinyl, imidazolidinyl, thiazolidinyl, oxadiazolyl, thiadiazolyl, morpholinyl, piperidinyl, piperazinyl, and pyrrolidinyl.

Each of the above substituents (alkyl, alkenyl, alkynyl, alkoxy, aryl, heteroaryl, or heterocyclyl) can be optionally substituted with one to three substituents as set forth in the embodiments recited above.

Methods for preparing the compounds of the present invention are illustrated in the following schemes and examples.

The first step for preparing an endothelin receptor antagonist involves the synthesis of a top piece substituent (4), ArX (X is halo) through the formation of tribromo ether (3) followed by treatment with a base as shown in Reaction Scheme A.

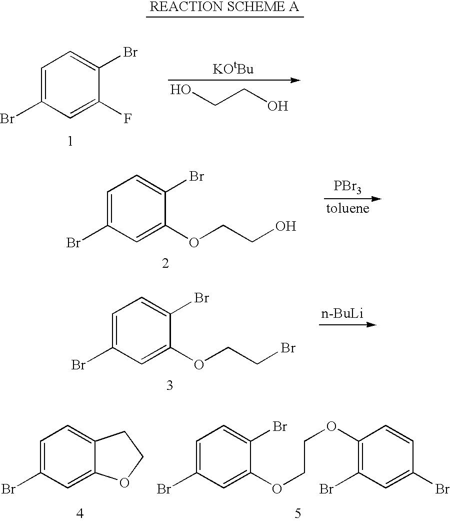

In Reaction Scheme A, ethylene glycol reacts with commercially available 1,4-dibromo-2-fluorobenzene (1) in the presence of potassium tert-butoxide to give the ether compound (2). The compound (2) is then converted to the tribromide (3) by treatment with a brominating agent (PBr₃) in an aprotic solvent such as toluene at a temperature between about 80° C. and about 90° C. The intermediates (2) and (3) can be used without purification. A small amount of water and additional PBr₃ (10 mol %) may be added in the middle of the reaction to improve the conversion rate of the compound (3) into the product (4) as shown in Table 1 (entries 3 and 4). Treatment of the tribromide (3) with n-BuLi or phenyllithium affords the desired 6-bromo-2,3-dihydrobenzofuran (4), which crystallizes in a mixture of methanol and water. The by-product (5) formed in the reaction can be removed by filtration.

TABLE 1
Temperature Effect on the Bromination Reaction

| | Temperature | % Conversion after |
|---|---|---|
| entry | ° C. | 4 hours |
| 1 | 25 | 46 |
| 2 | 80 | 90.5 |
| 3 | 90 | 92.6 |
| 4^{a} | 90 | 94.6 |

^{a}0.29 mol % water and 10 mol % PBr₃ were added after 2 hours at 90° C.

The α, β-unsaturated ester or amide

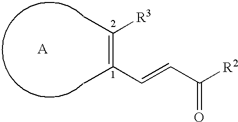

can generally be prepared in two steps:

1) a coupling reaction at the one position of ring A

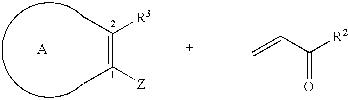

wherein R³ is CHO, Z is a leaving group such as Br, Cl, I, OTriflyl, OTosyl or Omesyl, and R² is OR⁴ or N(R⁵)₂; and

2) the conversion of the aldehyde (R³═CHO) to the desired chiral auxiliary (R³), wherein R represents

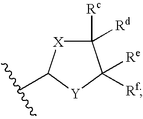

X and Y are independently O, S, or NR⁵; R⁴ is (C₁-C₈)-alkyl; R⁵ is (C₁-C₈)-alkyl or aryl; R^{c}, R^{d}, R^{e} and R^{f} are independently H, (C₁-C₈)-alkyl or aryl such that either R^{c} and R^{d} are not the same or R^{e} and R^{f} are not the same, or R^{c} and R^{e} or R^{d} and R^{f} can join to form a 5- or 6-membered ring, which is optionally substituted with one to three substituents selected from the group consisting of aryl, CO₂R⁴, CF₃, N(R⁵)₂, (C₁-C₈)-alkoxy, (C₁-C₈)-alkyl, (C₂-C₈)-alkenyl, (C₂-C₈)alkynyl, (C₃-C₈)cycloalkyl, CO(CH₂)ₙCH₃, and CO(CH₂)ₙCH₂N(R⁵)₂; and n is 0 to 5.

Reaction Scheme B below shows a method for the preparation of α, β unsaturated ester involving an amination, a formylation and a Heck reaction.

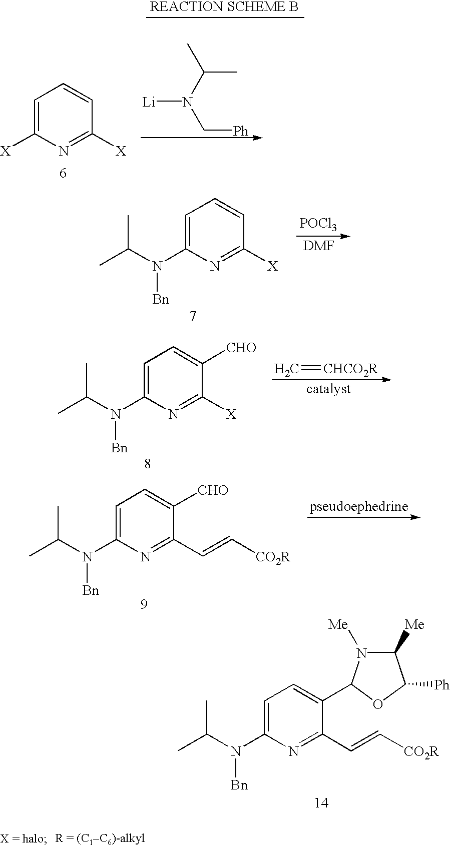

Commercially available disubstituted pyridine (6) is aminated by lithium N-isopropylbenzylamide to afford the compound (7). The aminated compound (7) was then regiospecifically formylated to give aldehyde compound (8) upon treatment with about 4 equivalents of POCl₃ in dimethylformamide (DMF) at a temperature range of about 35° C. to about 70° C. The aldehyde compound (8) then undergoes a Heck reaction with 1 to 5 equivalents of (C₁-C₆)-alkyl acrylate in the presence of an aprotic solvent, a base and a catalyst at a temperature range of 80° C. and 110C to provide the unsaturated ester (9) in high yield. The unsaturated ester (9) is then reacted with a chiral additive such as pseudoephedrine or N-methyl-cis-aminoindanol (not shown in the scheme) to give the protected aldehyde (16).

The aprotic solvent for a Heck reaction is selected from dimethylacetamide (DMAC), dimethylformamide (DMF), toluene and acetonitrile, and a base is selected from CH₃COONa, CH₃COONa.3H₂O and NaHCO₃. Preferred solvent and base are DMAC and CH₃COONa.3H₂O, respectively. Water may be added (about 6 equivalents) to the reaction mixture to enhance the reaction rate. For example, the reaction rate in the presence of CH₃COONa with water is 6 hours, whereas the reaction without water is 20 hours. The catalyst for the reaction is selected from PdCI₂(dppf)₂, PdCl₂ (PPh₃)₂, Pd(dba)₂, PdBr₂, Pd(OAc)₂, and (allyl)₂PdCl₂ dimer with tri-o-tolylphosphine. Preferred catalyst is PdCl₂(dppf)₂.

Another aspect of the invention involves the synthesis of a bottom piece (13), ArX (X is halo), of the compound according to Reaction Scheme C.

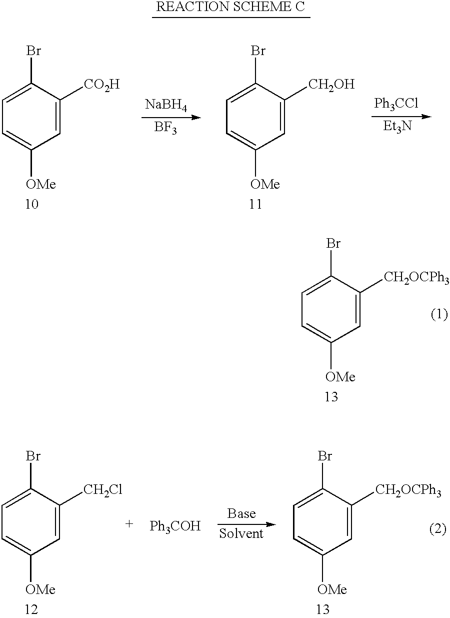

In Reaction Scheme C, the bottom piece of 2-Bromo-5-methoxybenzyl trityl ether (13) can be prepared either by a route (1) or a route (2). The route (1) involves a two-step synthesis via a reduction and a protection, whereas the route (2) provides a one-step synthesis by using commercially available benzyl chloride (12) in the presence of a base and an aprotic solvent. The base is selected from potassium tert-butoxide, KOH or NaH, and the solvent is selected from DMAC, DMSO or DMG. A mixture of potassium tert-butoxide and dimethyl acetamide (DMAC) is preferred. The compound (13) can be readily isolated by addition of water. As shown in Table 2 below, the optimal charge ratio of benzyl chloride (12):Ph₃COH:tert-BuOK is 1:1.1:1.05 with slow addition of the benzyl chloride.

TABLE 2
Preparation of 2-bromo-5-methoxybenzyl trityl ether (13)

| | Ph₃COH | 12 | t-BuOK | Addition of | Yield, 13 |
|---|---|---|---|---|---|
| Entry | (eq) | (eq) | (eq) | 12 | (% yield) |
| 1 | 1.1 | 1.0 | 1.05 | 1 h | 87% |
| 2^{a} | 1.1 | 1.0 | 1.05 | 1 h | 82% |
| 3 | 1.0 | 1.05 | 1.0 | 1 h | 82% |
| 4 | 1.1 | 1.0 | 1.05 | 5 min | 79% |

^{a}1% water is added to DMAC

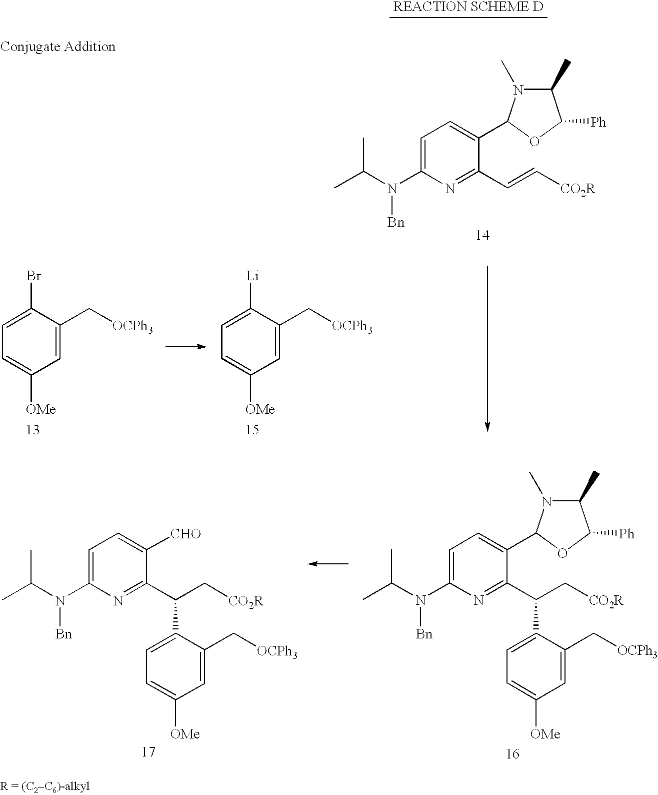

Compound (15) reacts with the cc, -unsaturated ester bearing a pseudoephedrine (14) or alternatively N-methyl-cis-aminoindanol chiral auxiliary, in an aprotic solvent or a mixture thereof (preferably THF/toluene) at a temperature of about −80° C. to about 0° C., preferably about −78° C. to about −50° C. Work up the reaction mixture with acid and water (to remove the auxiliary) at a temperature between about −15° C. and about 10° C. affords compound (17) in high yield and good selectivity. It is noted that other chiral axillary groups can be utilized in this asymmetric addition. (See WO 98/06698, published by the World Intellectual Property Organization on Feb. 19, 1998.)

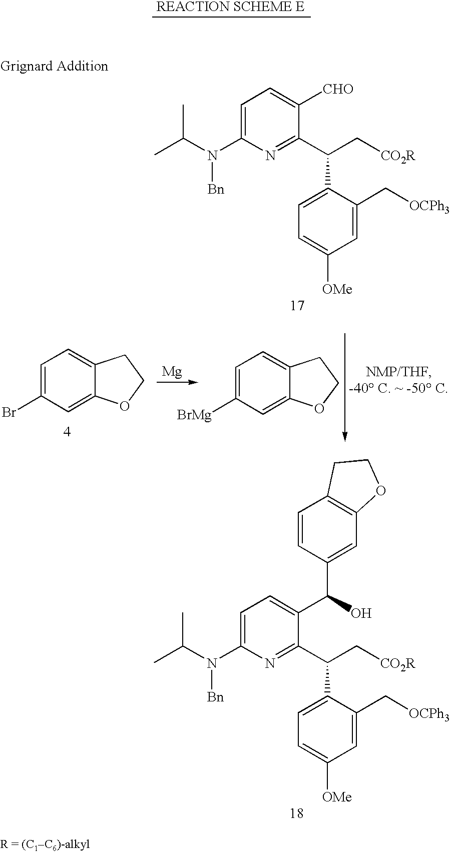

In Reaction Scheme E, addition of a Grignard reagent (prepared from the aryl bromide and magnesium) to the compound (17) in a mixture of TBF/NMP at about −80° C. to about 30° C. (preferably about

−40° C. to about −50° C.) affords compound (18) in quantitative yield and good diastereoselectivity. Addition of additives and/or selection of solvent may enhance the selectivity as shown in Tables 3, 4 and 5.

TABLE 3
The Effect of Additive on Grignard Addition of (17)
(R is tert-butyl)

| | Additive | | | | | |
|---|---|---|---|---|---|---|
| | MgBr₂. | | | | | |
| | Et₂O | LiBr | BF₃.Et₂O | ArLi | ZnCl₂ | DMPU |
| Selectivity | 7.6/1 | 6.7/1 | 5.3/1 | 1.8/1 | NR | 6.0/1 |

Adding about 2.5 equivalents of MgBr₂.Et₂O slows down the reaction but increases the selectivity to about 7.6/1. Similarly, about two equivalents of LiBr also slows down the reaction with slight increase of the selectivity (6.7/1, 50% conversion). Compared to MgBr₂.Et₂O and LiBr, addition of BF₃.Et₂O and DMPU (about 5 equivalents) results in low-conversion without much improvement in selectivity.

TABLE 4
The Effect of Solvent on Grignard Addition of (17)
(R is tert-butyl)

| | Solvent | | | | |
|---|---|---|---|---|---|
| | | | | | (1:1) |
| | THF | toluene | DMF | NMP | NMP/THF |
| T (° C.) | −78 | −78 | −60 to RT | −20 to −10 | −40 to −50 |
| Selectivity | 5/1 | 5.6/1 | NR | 15/1 | 25/1 |

As shown in Table 4, a non-polar solvent such as toluene fails to improve the selectivity (5.6/1), whereas a polar solvent such as N-methylpyrrolidineone (NMP) considerably enhances the selectivity (15/1). A mixed solvent of (1:1) NMP:TH at about −40° C. to about −50° C. even further enhances the selectivity resulting a cleaner reaction with improved stereoselectivity (25/1).

TABLE 5
The Effect of Solvent on Grignard Addition of (17)
(R is isopropyl)

| | | | | (1:1) |
|---|---|---|---|---|
| Solvent | THF | NMP | NEP | NMP/THF |
| T (° C.) | −78 | −20 to −10 | −50 to 25 | −40 to −50 |
| Selectivity | 3.8/1 | 22/1 | NR | 35/1 |

As shown in Table 5, the selectivity of the Grignard addition to aldehyde compound (17) where R is isopropyl, in THF is very low (3.8/1). In NMP, the selectivity improves to about 22/1 at a temperature about −20° C. to about −10° C., but large amounts of a side product is observed. A mixed solvent of (1:1) NMP:THF at a temperature of about −40° C. to about −50° C. significantly enhances the selectivity resulting a cleaner reaction with a higher stereoselectivity (35/1).

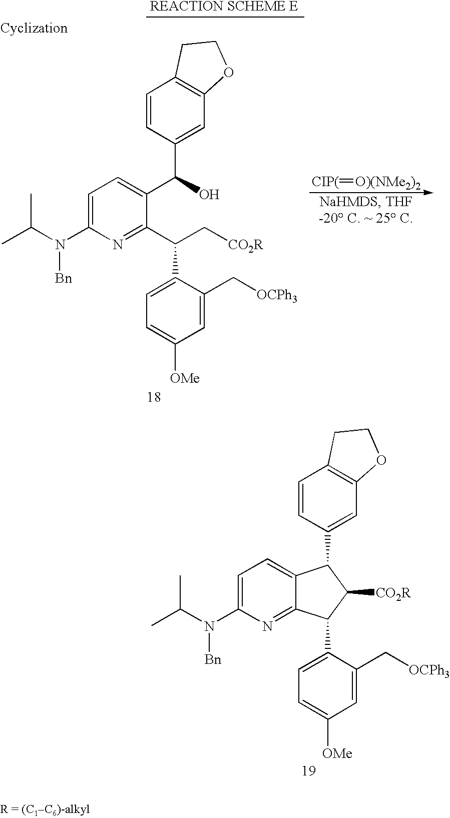

In Reaction Scheme F, cyclization of a Grignard addition compound (18) by treatment with about 1 to 2 equivalents of N,N,N′,N′-tetramethylphosphorodiamidic chloride, [(CH₃)₂N]₂POCl, and about 1 to 6 equivalents (preferably 4 to 5 equivalents) of sodium hexamethyldisilazide (NaHMDS) or LiHMDS in an aprotic solvent at about −80° C. to about 30° C. (preferably about −20° C. to about 25° C.) affords a cyclized compound (19). Preferred aprotic solvents are THF, toluene and a mixture of THF/toluene. A reaction in NaHMDS and THF are preferred.

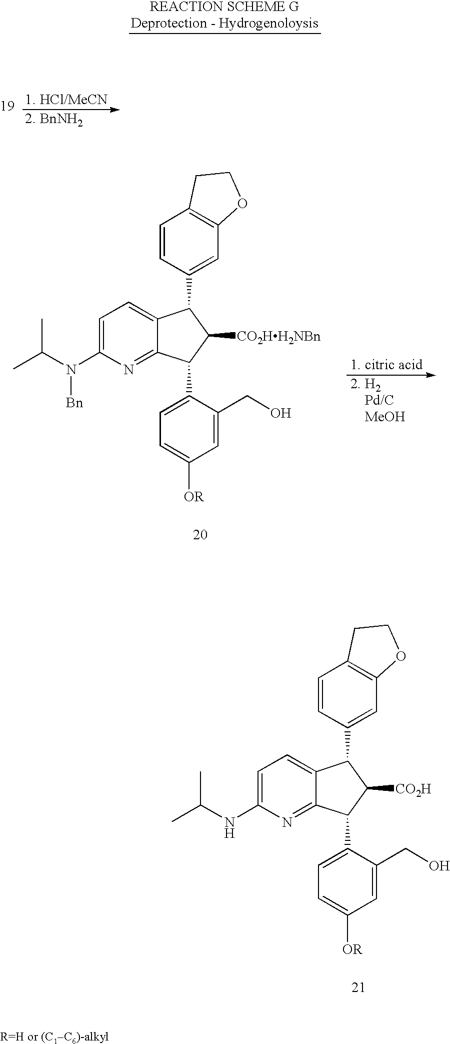

In Reaction Scheme G, deprotection of the compound (19) by removing protecting groups with concentrated HCl in acetonitrile at a temperature about 0° C. to about 25° C. followed by crystallization of its benzylamine salt affords the penultimate intermediate (20) in quite high yield and purity. When alkyl substituent is isopropyl in compound (19), deprotection occurs after treating the reaction mixture with MsOH, MeOH and then NaOH (aq) at a temperature about 40° C. Salt breaking in citric acid followed by hydrogenation of the benzylamine salt (20) over palladium under hydrogen (about 40 psi) in a protic solvent at a temperature range of about 25° C. to about 40° C. affords the desired product of carboxylic acid (21) in high yield. The protic solvent is selected from methanol, ethanol, isopropyl alcohol (IPAc), methanol/TBF and methanol/DMF. Methanol is a preferred solvent. Addition of THF or DMF may be necessary to remove the catalyst after the hydrogenation. Work up of the reaction mixture followed by crystallization in methanol, THF/water or DMF/water affords the desired compound (21) in high yield (90-95% yield).

The following examples illustrate the preparation of the compound of Formula I and as such are not to be considered as limiting the invention set forth in the claims appended hereto.

### EXAMPLE 1

1,4-Dibromo-2-hydroxyethoxybenzene (2)

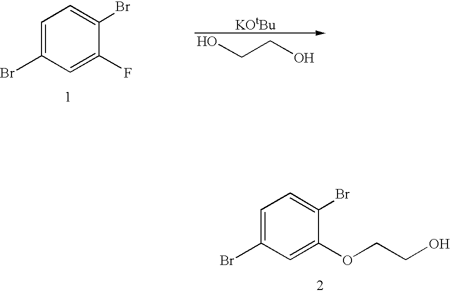

Under nitrogen, to a three-necked flask is added ethylene glycol (350 mL), 1,4-dibromo-2-fluorobenzene, 1 (68.6 g, 270 mmol) and 1-methyl-2-pyrrolidinone (35 mL). Solid potassium tert-butoxide (112 g, 950 mmol) is added over 5 minutes. The batch is heated to 97° C. to 100° C. and aged at the same temperature for 8 hours until HPLC indicated <1.0% of starting material. The batch is then allowed to cool to about 24° C., and water (137 mL, 2 mL/g 1) is added over 0.5 hour. The mixture is filtered, and the solid is washed with ethylene glycol. About 1.2 L of water is added to the combined filtrate, which is wash for over 30 minutes. The mixture is then cooled to about 15° C. and aged for about an hour. The solid is collected by filtration, washed with water, and dried by suction under nitrogen. Alcohol product 2 is isolated as a light yellow solid (69.6 g, 87% yield, 100 A% pure).

HPLC Conditions:

Zorbax RX-C18, 4.6×250; MeCN/0.1% H₃PO₄; 1.5 mL/min; UV detector at 220 nm; Retention times (min): 1,4-dibromo-2-fluorobenzene 1 (9.6), 1,4-dibromo-2-hydroxyethoxybenzene 2 (5.4) and dimer 5 (13.8).

### EXAMPLE 2

2-Bromoethoxy-1,4-dibromobenzene (3)

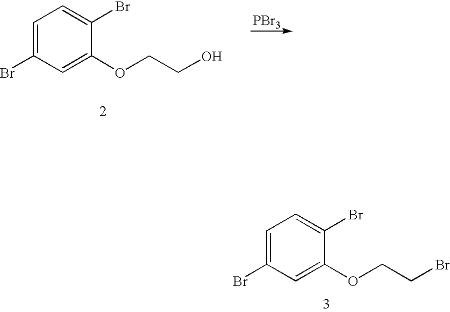

To a solution of 1,4-dibromo-2-hydroxyethoxybenzene (10.05 g, 33.9 mmol) in toluene (72 mL) is added PBr₃ (1.45 mL, 15.27 mmol). The mixture is heated to about 90° C. and aged for about two hours. The remainder of the PBr₃ is added followed by water. The batch is heated at about 90° C. for an additional 8 hours and then cooled to room temperature. The batch is slowly quenched with 60 mL of 1N NaOH for about 30 minutes. The two layers are separated. The organic layer is washed with water and saved for the next step.

HPLC Conditions:

Zorbax RX-C18, 4.6×150; MeCN/0.1% H₃PO₄ at 1.0 mL/min; UV detector at 230 nm; Retention times (min): 2-bromoethoxy-1,4-dibromobenzene 3 (10.5 min)

### EXAMPLE 3

6-Bromo-2,3-dihydrobenzofuran (4)

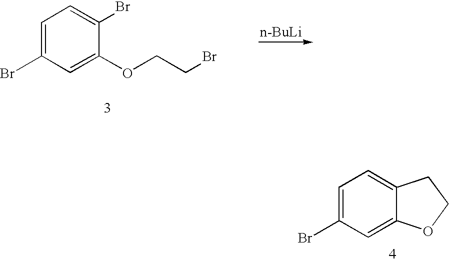

The tribromide solution from the previous step is concentrated to about 10 L and flushed with 20 L of dry toluene. The final volume is about 8 L before the addition of 18 L of THF. The batch is cooled to about −73° C. and n-butyllithium (1.6M in hexane, 6.0 L) is added slowly, keeping the temperature <−70° C. The starting material is assayed by IPLC for 15 minutes after the completion of the addition, and then more n-butyllithium is added (a total of 0.4 L) until no starting material is detected by HPLC. Excess n-butyllithium is quenched with acetic acid before the batch is allowed to warm to 0° C. About 17 L of water is added and the two layers are separated. The organic layer is washed with 0.5 N NaOH and water. The batch is concentrated to about 8 L and flushed with methanol. The final volume is adjusted to about 8 L and the batch is cooled to about 15° C. to about 20° C. until some product crystallized, whereupon about 7 L of water is added over two hours (final methanol:water is about 1:1). The batch is aged at about 15° C. for about an hour and then filtered. The solid is washed with 2:3 methanol:water and dried by suction under nitrogen for about six hours. Product 4 is isolated as a white solid (1.71 kg, KF =7.3μg/Ig, 95.07 A%, 97 wt %, and 85.5% corrected yield).

HPLC Conditions:

Zorbax SB-C8 4.6×250; MeCN/0.1% H₃PO₄ at 1.5 mL/min; UV detector at 220 nm; Retention times (min): 6-bromo-2,3-dihydrobenzofuran 4 (7.4).

### EXAMPLE 4

Mono-amination of 2,6-Dibromopyridine (6)

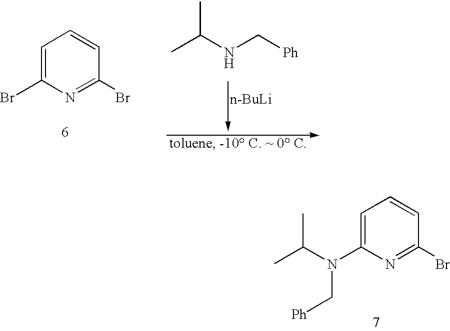

n-BuLi (1.27 L, 2.5M, 3.18 mol) is added to a solution of N-isopropylbenzylamine (473 g, 3.17 mol) in 0.67 L toluene and 0.72 L hexane at −15° C. to −10° C. for about two hours. The mixture is aged at −10° C. to 0° C. for 0.5 hour to give the lithium amide. It is then transferred into a slurry of 2,6-dibromipyridine (500 g, 2.11 mol) and N-isopropylbenzylamine (317 g, 2.11 mol, 1.0 equiv.) in toluene (2.5 L) and hexane (2.5 L) at 5° C. to 10° C. for about an hour. The mixture is stirred at 0° C. until the reaction is completed as monitored by HPLC. The reaction is quenched by transferring the reaction mixture via a cannula into 2N HCl (2.5 L) at 10° C. to 20° C. with vigorous stirring. The flask is rinsed with hexane. About 1.5 L of DMF is added to dissolve most of the dark precipitates. The mixture is stirred for 20 minutes. The layers are separated, and then the organic layer is washed with a mixture of 3:1 DMF:water and water. It is concentrated under vacuum (100 to 40 mmHg, 40° C. bath) to a minimum volume, flushed with toluene (40 to 20 mmHg, 40° C.˜50° C. bath), and then pumped for two hours to give the crude product 7 (596 g, 93.3 w%, 86% yield). ¹H NMR indicated 5.7 w% toluene. HPLC indicated 2.7 A% toluene, 0.8 A% bis-amination product and 94.4 A% of the desired product 7.

HPLC Conditions:

Zorbax SB-C8 4.6×250 mm; MeCN 40-90% in 15 min; 1.50 mL/min, 10 mM Trizma buffer (pH=7); 30° C., UV detection at 220 nm; Retention times (min): 2,6-dibromopyridine 6 (5.8),

N-isopropylbenzylamine (5.1, broad); toluene (6.7), 2-(N-isopropylbenzylamino)-6-bromopyridine 7 (12.6), and bisamination (16.7).

### EXAMPLE 5

Formylation

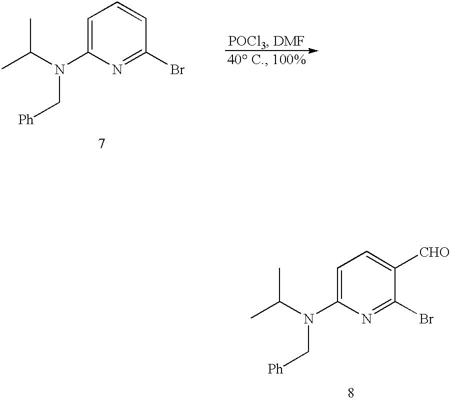

A solution of crude 2-(N-isopropylbenzylamino)-6-bromopyridine 7 (550 g, 93.3 w%, 1.68 mol) in DMF (2.8 L) is cooled to 10° C., and then POCl₃ (670 mL, 1.10 Kg, 7.2 mol, 4.3 equiv.) is added by using a dropping funnel while maintaining the batch temperature below 30° C. for about 1.2 hours. The mixture is heated to about 40° C. and then aged overnight for about 15 hours. Once the reaction is completed, the reaction mixture is cooled to below 20° C. and cannulated into a mixture of water and toluene with vigorous stirring and ice-water cooling to maintain <20° C. for about two hours. After separating the layers, the aqueous DMF layer is extracted with more toluene. The combined toluene layer is washed with water and then treated with Darco-KB (50 g) for about 0.5 hour. The mixture is then filtered through a Solka-Floc pad and the filter pad is washed with toluene. The filtrate is concentrated under vacuum (about 40° C.˜50° C. bath, 30-50 mmHg), and the residue is pumped under high vacuum overnight to give the crude product 8 as a brown oil (570 g, 102% yield uncorrected for purity).

### EXAMPLE 6

Heck Reaction:

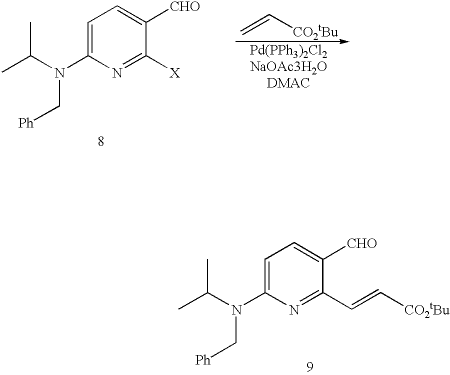

A 2 L three-neck round bottom flask equipped with a mechanical stirrer, temperature probe, and nitrogen inlet is charged with a degassed solution of bromoaldehyde 8 in dimethylacetamide. The reaction is purged with nitrogen for about 20 minutes. Both sodium acetate trihydrate (NaOAc.3H₂O ) and t-butyl acrylate are added to the solution. Finally the Pd catalyst is added to the reaction vessel, and the vessel is flushed with nitrogen. The resulting mixture is stirred mechanically for about 9 hours at 80° C. When the reaction is completed, the solution is cooled to room temperature, diluted with toluene (7.5 ml/g of starting material) and filtered through solka floc. The solka floc is then washed with 2.5 ml/g of toluene. The solution is washed once with water. The organic layer is azeotroped with toluene, and the material is taken into the next step at a final volume of 620 mL.

HPLC Conditions:

Waters Symmetry C8, 4.6 mm×250 mm; TSP UV2000 dual wavelength, 1 AU/volt output; Acetonitrile; 45° C.; 1.5 ml/min.; UV detection at 220 nm; Retention times (min.): aldehyde 8 (X =Br, 10.8; X =Cl, 10.3), cis Heck isomer (11.2), and trans Heck isomer (13.4).

### EXAMPLE 7

2-Bromo-5-methoxybenzyl trityl ether:

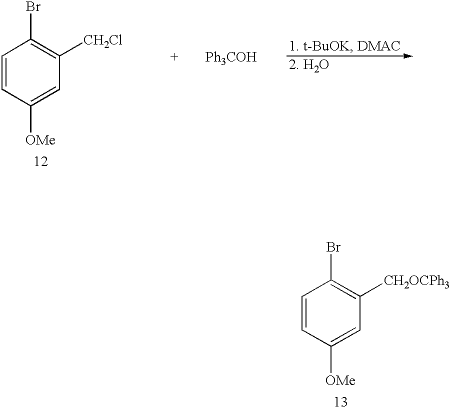

Under nitrogen, dimethylacetamide, (DMAC, 3.14 L), Ph₃COH (573 g, 2.2 mol) and tert-BuOK (236 g, 2.1 mol) are sequentially added to a three-necked 12 L flask, and then 2-bromo-5-methoxybenzyl chloride 12 (470 g, 2.0 mol) in DMAC (0.66 L) is added over an hour. The reaction mixture is stirred at room temperature for another hour. About 1.26 L of water is slowly added to the reaction mixture over an hour to crystallize the product. The slurry is stirred at room temperature for another hour and then filtered. The wet cake is washed with about 3 L of 80:20 DMAC:H₂O and water. The cake is dried by vacuum suction under nitrogen for 12 hours to give the compound 13 (800 g, 99.5 W%, 99.7 A%) as a bright white crystalline solid. The use of 80:20 DMAC:H20 is recommended for washing to remove by-products formed in the reaction. The additional wash with water can remove inorganic substances, such as KCl. By-products, dibenzylether and stilbene, formed in the reaction can also be removed by crystallization.

HPLC Conditions:

Zorbax RX-C8, 4.6×150; MeCN/H₂O at 1.5 mL/min; UV detector at 220 nm; Retention times (min): Ph₃COH (8.5), dibenzyl ether (12.5), stilbene (13.6), and trityl ether 13 (16.4).

### EXAMPLE 8

N,O-Acetal Formation:

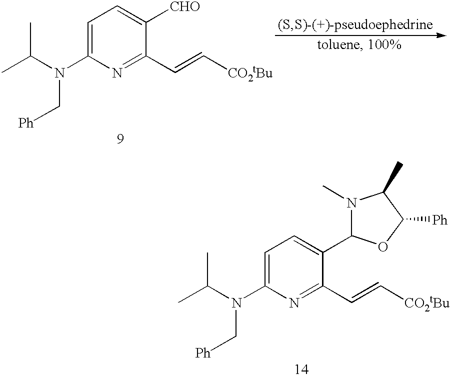

A 3 L three-neck round bottom flask equipped with a mechanical stirrer, nitrogen line, Dean-Stark trap with condenser and temperature probe is charged with toluene (0.93 L, KF=52 μg/nl) and the Heck product 9 (185.8 g). To the solution, (S,S)-pseudoephedrine (104.1 g) and camphorsulfonic acid (csa, 2.7 g) are added. The reaction mixture is then refluxed vigorously until 9 is completely consumed. Upon cooling the mixture to about room temperature, Florisil (93 g) is added and the slurry is stirred for about 30 minutes. The Florisil is then filtered off and washed with toluene. The filtrate and wash are combined and washed with water. The organic layer is concentrated to about 1.7 L. The solution is flushed with toluene until the KF is 250 μg/ml.

### EXAMPLE 9

Conjugate Addition:

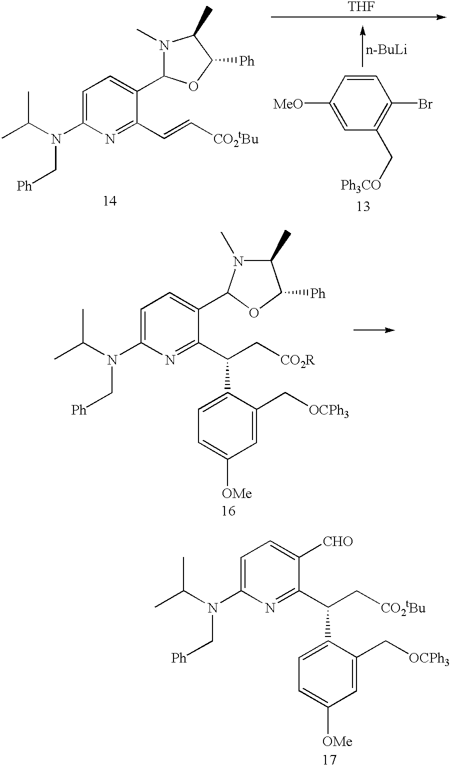

A 12 L three-neck round bottom flask equipped with a mechanical stirrer, nitrogen line and temperature probe is charged with aryl bromide 13. The flask is then purged with nitrogen. Degassed toluene (2.1 L, KF=84μg/mL) and THF (2.1 L, KF=278μg/mL) are then charged, and the flask is purged with nitrogen. The solution is cooled to about −70° C. and 1.6M nBuLi (537 mL) is added by using a gas tight syringe over 25 minutes. The solution is aged for 15 minutes and then checked by HPLC for residual ArBr. When ArBr is completely consumed, a solution of 14 in about 1.7 L toluene is added to the reaction mixture via canula over 20 minutes. The reaction mixture is aged for about 25 minutes, and then warmed to about −50° C. and quenched by the addition of HOAc (179 mL). The mixture is again allowed to warm to about 0° C. Aqueous citric acid (333 g citric acid +930 mL water) is added, and the biphasic mixture is stirred at room temperature for 16 hours. The mixture is then transferred to a separatory funnel and the aqueous layer is removed. The organic layer is washed twice with saturated aqueous NaHCO₃, and once with water. The organic layer is assayed and concentrated to about 1.3 L by removing the solvent in preparation for the Florisil treatment. A large sintered glass funnel is packed with a slurry of Florisil (2.58 kg) in 30% MTBE in toluene (2.5 L). The toluene solution of 17 is charged to the top of the Florisil plug, and the material is eluted with 30% MTBE in toluene. About 2.4 L of solution (containing no product) is collected and discarded. An additional 10 L of solution is collected and assayed for 17. The combined fractions containing product are concentrated and azeotropically dried to afford 350.3 g of 17 (95% recovery from the florisil treatment). The material is carried forward into the Grignard addition.

HPLC Conditions:

Waters Symmetry C8, 4.6×250 mm; TSP UV 2000 dual wavelength, 1 AU/volt output; acetonitrile or (1:1) acetonitrile:water; 1.0 ml/min; UV detector at 220 nm; 50:50 ACN:water; Retention time at room temperature (min): 9 (19.5), 14 (25.4), 13 (21.4), 16 (40.8) and 17 (27.0).

### EXAMPLE 10

Grignard Addition:

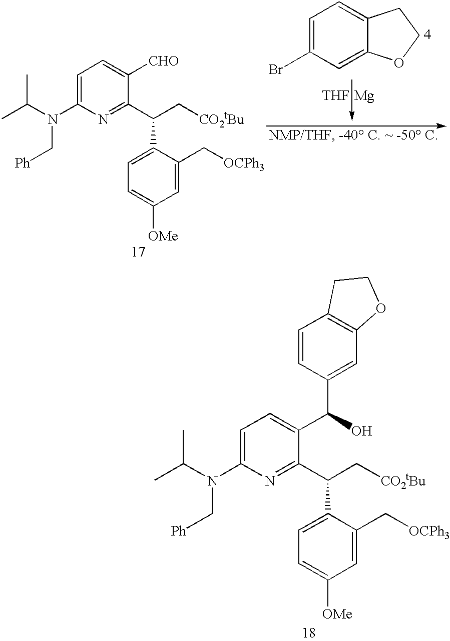

Step 1: Drying of ArBr (4):

A solution of ArBr 4 (about 300 g, containing 2 to 4 w% water) in THF (600 mL) is stirred with 60 g of molecular sieves overnight. The spent molecular sieves is removed and rinsed with 50 mL of TBF. Another 60 g of fresh molecular sieves is added and the mixture is stirred for about 5 hours (KF of the TBF solution is approximately 100μg/mL). The spent molecular sieves is removed and rinsed with THF (50 mL). Another 30 g of fresh molecular sieves is added to the combined THF solution. Upon stirring for about 2 hours, assay of the solution indicates that it contains 322g/L of the compound 4.

Step 2: Grignard Preparation:

To a 2 L three-neck round-bottom flask equipped with an efficient condenser, a thermocouple thermometer and a mechanical stirrer is added Mg (27.2 g, 1.12 mol) and THF (650 mL). The ArBr 4 solution in TBF (635 mL, 322g/L, 204.5 g, 1.03 mol) is charged into the dropping funnel. The system is degassed by vacuum/N₂ cycle three times and then the mixture is heated to about 50° C. A portion of the ArBr 4 solution (about 50 mL) is added and the mixture is stirred until the reaction is initiated. The remaining ArBr solution is added at between 50° C. and 60° C. for about 2 hours. The mixture is aged at 50° C. for about an hour to give a solution of Grignard reagent ArMgBr.

HPLC Conditions:

Zorbax SB-C8 4.6×250 mm; MeCN 40%90% in 15 min; 1.50 mL/min, 10 mM Trizma buffer (pH=7); 30° C., UV detection at 220 nm; Retention times (min): ArBr 4 (7.4) and ArH (5.3).

Step 3: Grignard Addition:

To a 5 L four-neck round-bottom flask equipped with a mechanical stirrer, a thermocouple thermometer and a nitrogen inlet is charged with dry crude conjugate addition product 17 (514 g) (assay 258 g), NMP (1.25 L) and TMF (0.75 L). The mixture is degassed by vacuum/N₂ cycle three times and then cooled to −50° C. Approximately 1.1 L of the Grignard reagent is charged via a cannula in an hour at about −45° C. to −50° C. The mixture is aged for an hour at about −50° C. BPLC is used to monitor the completion of the reaction. More ArMgBr may be added if necessary. The reaction is quenched by cannulating the reaction mixture into an aqueous NU4Cl (1.7 L 15 w%) with stirring for about 40 minutes. Toluene is added to aid the layer separation. The organic layer is then washed with NH₄Cl (15 w%, 0.5 L×2) and brine (IL) and then concentrated to a minimum volume (about 0.8 L). It is then dried by flushing with more toluene (final weight after the flush is 744 g). BPLC assay indicates the presence of 294 g of the product 18 (98% yield) in the residue. The diastereoselectivity is about 96/4.

HPLC Conditions:

Zorbax SB-C8 4.6×250 mm; MeCN 60%95% in 15 min; 1.50 mL/min, 10 mM Trizma buffer (pH=7); 30° C., UV detection at 220 nm; Retention times (min): conjugate addition product 17 (18.0) and Grignard addition product 18 (18.7).

Normal Phase HPLC Conditions for Diastereoselectivity Measurement:

YMC PVA 4.6×250 mm; hexane:IPAc (95:5); 1.00 mL/min; UV detection at 220 nm; Retention times (min): major isomer (9.1) and minor isomer (7.4).

### EXAMPLE 11

Cyclization:

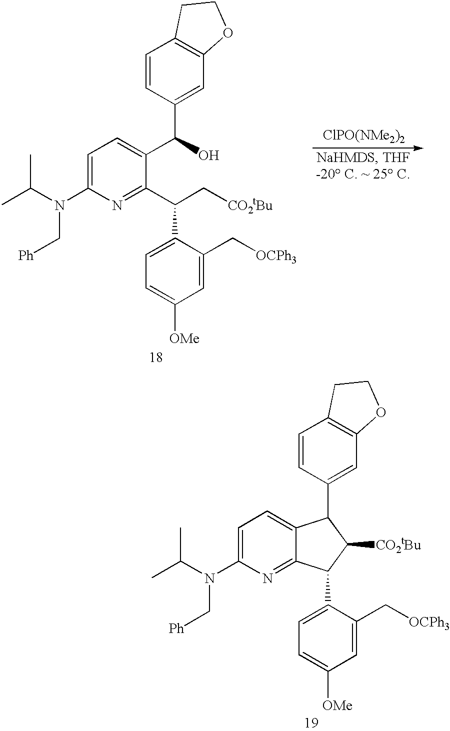

To a 5 L four-neck round-bottom flask equipped with a dropping funnel, a mechanical stirrer, a thermocouple thermometer and a nitrogen inlet is added the crude Grignard addition product 18 (780 g, 295 g assay) and THF (1.2 L). The system is degassed by vacuum/N₂ cycle and then cooled to −20° C. CIP(O)(NMe₂)₂ (74 mL, 0.5 mol, 1.5 equiv.) is added followed by slow addition of N S (1.67 L, 2 hours) at about −20° C. to 0° C. by a dropping funnel. The mixture is then aged for 3 hours at 0° C. and the completion of the reaction was confirmed by HPLC (<1 A% SM). Additional amount of CIP(O)(NMe₂)₂ (0.1 equiv.) and NaHMDS (0.2 equiv.) may be added if necessary. The reaction is quenched by slowly adding about 600 mL of water followed by slow addition of 400 mL of acetic acid. The mixture is stirred for about 0.5 hour at 15° C. to 25° C., and then the layers are separated. The organic layer is washed with 1.0 L of (1:1) brine:water and then 1.0 L of brine. It is concentrated under reduced pressure (30˜60 mmHg, 40° C. bath) to 666 g and then flushed with 660 mL of MeCN (90˜40 mmHg, 40° C. bath). The crude product 19 is used directly for the deprotection step.

### EXAMPLE 12

Deprotection and Benzylamine Salt Formation:

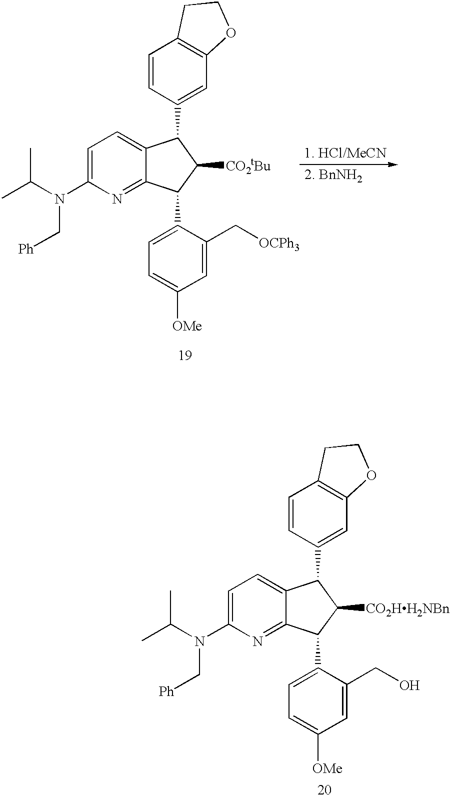

To a 5 L three-neck round-bottom flask equipped with a mechanical stirrer, a thermocouple thermometer and a dropping funnel is charged with about 2 L of MeCN. The mixture is cooled to 0° C. and then 900 mL of concentrated HCl is added by a dropping funnel at <15° C. The crude cyclization product (625 g crude, about 250 g pure) is diluted with 400 mL of MeCN and then charged into the HCl in MeCN solution at 5° C. to 15° C. The starting material flask is rinsed with additional amount of acetonitrilec. The mixture is allowed to warm to 20° C. and stirred overnight. The completion of the deprotection is confirmed by HPLC (<2% t-butyl ester intermediate).

HPLC Conditions:

Zorbax SB-C8 4.6×250 mm; MeCN 30-80% in 15 min; 1.50 mL/min, pH=7, 10 mM Trizma buffer; 30° C., UV detection at 220 nm; Retention times (min): t-butyl ester intermediate (17.9), deprotection product (9.1), and trityl alcohol (12.2).

The mixture is then cooled to 0° C. and neutralized with NaOH (10N, 1.16 L at <25° C.) until the pH of the aqueous layer is between 5 and 7. Water (500 mL) is added to dissolve the precipitated inorganic salt after neutralization. About 1 L of MTBE is added and the mixture is stirred for 15 minutes. The mixture is then allowed to settle for about 20 minutes and the layers are separated. The organic layer is extracted with NaOH. Assay of the organic layer indicates about 1% to 2% product loss. The combined aqueous layer is back extracted with MTBE and the back extract is then washed with 0.1N NaOH. About 1.5 L of MTBE is added to the combined NaOH extracts, and then the mixture is neutralized with 2N HCl to pH of about 5 to 6. The organic layer is separated and then washed with brine. The brine washes are combined with the aqueous layer and then extracted with 1 L of IPAc. The organic layer is washed with brine. The combined organic layer is concentrated to a minimum volume of about 0.4 L and flushed with IL of IPAc. The residue is diluted with isopropyl alcohol (IPAC) and treated with about 10 g of Darco-KB for 2 hours. The mixture is then filtered through a Solka-Floc pad. The pad is rinsed with IPAc. Assay of the filtrate indicated the presence of 175 g (77% overall yield from Michael addition) of the product as its benzylamine salt equivalent. It is concentrated to 844 g and then 15 mL of benzylamine and 1 g of seed are added. The mixture is then stirred under nitrogen for 3 hours. The remaining benzyamine is added slowly for an hour, and then the mixture is stirred overnight at room temperature. The product is collected by filtration and the filter cake is washed with IPAc until the wash becomes almost colorless. The product is dried by sucking air through it for about 3 hours until constant weight is obtained to give 158 g of the benzylamine salt 20 (97.3 A%, 70% overall yield from Michael addition). Mother liquor loss is 18 g (8.0%).

### EXAMPLE 13

Hydrogenolysis of Benzlamine Salt (20):

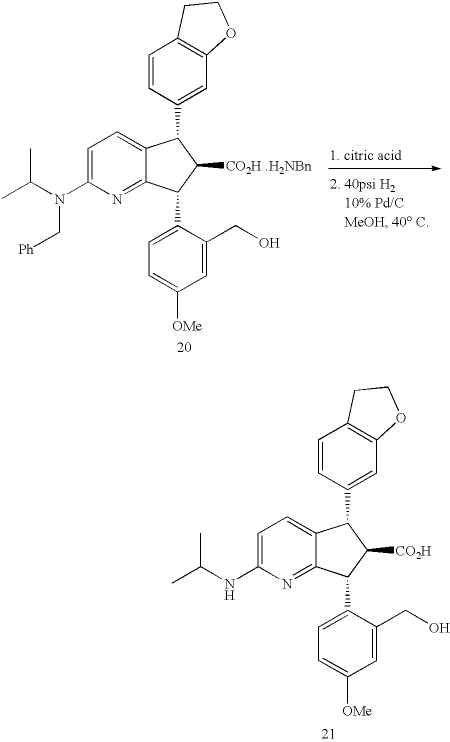

To a slurry of the benzylamine salt 20 (70 g, 96 w%, 0.10 mol) in MTBE (750 mL) is added aqueous citric acid (500mL 0.25M). The mixture is stirred until all solids were dissolved. The pH of the aqueous layer is about 3 to 5. The layers are then separated, and the organic layer is sequentially washed with 0.13M aqueous citric acid, water and brine. The organic layer is concentrated under reduced pressure of about 200 mmHg at 30° C. bath and flushed with 400 mL of methanol. The residue is diluted with methanol and submitted to the hydrogenolysis (5.64 g 10% Pd/C, 40psi, 40° C., 3 hours). The completion of the reaction is confirmed by HPLC. The reaction mixture is then diluted with 700 mL of THF to dissolve the product and then filtered through a Solka-Floc pad to remove the Pd catalyst. The pad is rinsed with 500 mL of 2:1 THF:MeOH mixture. The filtrate is concentrated and then flushed with methanol. The residue is diluted with 500 mL of methanol and the slurry is stirred at 40° C. for 0.5 hour and then aged at room temperature overnight. The product is collected by filtration and the filter cake is washed with methanol. It is dried by sucking air through it until a constant weight is achieved to afford the final product as a white solid (95.3% yield, >98 A%).

HPLC Conditions:

Zorbax SB-C8 4.6×250 mm; MeCN 10-70% in 15 min; 1.50 mL/min, 0.1% H₃PO₄; 30° C., TV detection at 220 nm. Retention times (min): benzylamine salt 20 (13.7) and the final compound 21 (9.8).

What is claimed is:

1. A process for preparing a compound of Formula I, 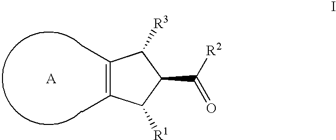
wherein: 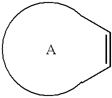
represents:
(a) 5- or 6-membered heterocyclyl containing one to three double bonds, but at least one double bond and 1 to 3 heteroatoms selected from O, N and S, and the heterocyclyl is optionally substituted with one to three substituents selected from the group consisting of: OH, CO R⁴, Br, Cl, F, I, CF, N(R⁵)₂, (C₁-C₈)-alkoxy, (C,-C₈)-alkyl, (C₂-C₈)-alkenyl, (C₂-C₈)-alkynyl, (C₃-C₈)-cycloalkyl, CO(CH₂)CH₃, and CO(CH₂)C₂(R⁵)₂;
(b) 5- or 6-membered carbocyclyl containing one or two double bonds, but at least one double bond, and the carbocyclyl is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂N(R⁵)₂; or
(c) aryl, wherein aryl is defined as phenyl or naphthyl, which is optionally substituted with one to three substituents selected from the group consisting of: OH, CO₂R⁴, Br, Cl, F, I, CF₃, N(R⁵)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂, or when aryl is substituted on adjacent carbons they can form a 5- or 6-membered fused ring having one to three heteroatoms selected from O, N, and S, this ling being optionally substituted on carbon or nitrogen with one to three substituents selected from the group consisting of: H, OH, CO₂R⁶, Br, Cl, F, I, CF₃, N(R⁷)₂, (C₁-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;
and wherein (C₁-C₈)-alkoxy, (C₁-C₈)-alkyl, (C₂-C₈)-alkenyl, (C₂-C₈)-alkynyl, or (C₃-C₈)-cycloalkyl substituent of aryl is further optionally substituted with one to three substituents
selected from the group consisting of: OH, CO₂R⁴, Br, Cl, F, I, CF₃, OCPh₃, N(R⁵)₂, (C₁-C₈)-alkoxy, (C₃-C₈)-cycloalkyl, CO(CH₂)ₙCH₃, and CO(CH₂)ₙCH₂N(R⁵)₂;
R¹ is:
(a) (C]-C₈)-alkyl, (C₂-C₈)-alkenyl, (C₂-C₈)-alkynyl, or (C₃-C₈)-cycloalkyl,
(b) aryl, wherein aryl as defined above, or
(c) heteroaryl, wherein heteroaryl is defined as a 5- or 6-membered aromatic ring containing one to three heteroatoms selected from O, N and S, and is optionally substituted with one to three substituents selected from the group consisting of: OH, CO₂R⁴, Br, Cl, F, I, CF₃, N(R⁵)₂, (C₁-C₈)-alkoxy, (C,-C₈)-alkyl, (C₂-C₈)-alkenyl, (C₂-C₈)-alkynyl, (C₃-C₈)-cycloalkyl, CO(CH₂)ₙCH₃, and CO(CH₂)ₙCH₂N(R⁵)₂;
R² is: OR⁴ or N(R⁵)₂;
R³ is:
(a) (C₁-C₈)-alkyl,
(b) (C₂-C₈)-alkenyl,
(c) (C₂-C₈)-alkynyl,
(d) (C₃-C₇)-cycloalkyl,
(e) aryl, wherein aryl as defined above,
(f) heteroaryl, wherein heteroaryl as defined above,
(g) —CHO,
(h) —CO—(C₁-C₈)-alkyl,
(i) —CO-aryl,
(j) —CO-heteroaryl, or
(k) —CO₂R;
n is: 0 to 5;
t is: 0,1 or 2;
R⁴ is: H, or (C₁-C₈)-alkyl;
R⁵ is: H, (C₁-C₈)-alkyl or aryl, wherein aryl as defined above;
R⁶ is: H, (C₁-C₈)-alkyl or aryl, wherein aryl as defined above; and
R⁷ is: H, (C₁-C₈)-alkyl, aryl or alkyl, wherein aryl is optionally substituted with one to three substituents selected from the group consisting of: OH, CO₂R⁴, Br, Cl, F, I, CF₃, N(R⁵)₂, (C,-C₈)-alkoxy, (C₁-C₈)-alkyl, (C₂-C₈)-alkenyl, (C₂-C₈)-alkynyl, (C₃-C₈)-cycloalkyl, CO(CH₂)ₙCH₃, and CO(CH₂)₁₁CH₂N(R⁵)₂, or when two R substituents are on the same nitrogen they can join to form a ring of 3 to 6 atoms;
comprising the steps of:
(1) reacting a Grignard reagent with a conjugate adduct compound of Formula II, 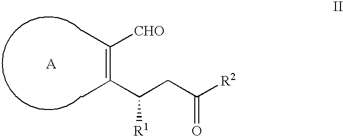
in the presence of a first aprotic solvent and optionally an additive at a temperature range of about −80° C. to about 30° C. to give a Grignard addition product of Formula III; and 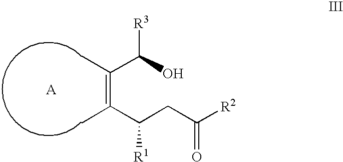
(2) adding phosphoramide reagent to a mixture of the Grignard addition product of Formula III, a second aprotic solvent and a base at a temperature range of about −80° C. to about 30° C. to produce the desired compound of Formula I.

2. The process of claim 1, wherein the first or second aprotic solvent is selected from the group consisting of tetrahydrofuran, acetonitrile, dimethylacetamnide, dimethylformamide, diethyl ether N-methylpyrrolidinone, dichloromethane, methyl t-butyl ether, toluene, benzene, hexane, pentane, dioxane, and a mixture thereof.

3. The process of claim 2, wherein the first aprotic solvent is a 1:1 mixture of N-methylpyrrolidinone and tetrahydrofuran at temperature range of about −40° C. to about −50° C. or N-methylpyrrolidinone at temperature range of about −20° C. to about −10° C.

4. The process of claim 3, wherein the additive is selected from the group consisting of MgBr₂.Et₂O, LiBr, BF₃.ET₂O, ArLi, and DMPU.

5. The process of claim 4, wherein the Grignard reagent is ArMgX, which is prepared from ArX and Mg.

6. The process of claim 5, wherein ArX is prepared by the following steps:
(a) reacting 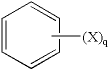
with HO(CH₂)ₘOH in the presence of a base to give 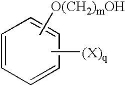
wherein q is 1 to 5, m is 2, 3, or 4, and X is Br, Cl, F, or I;
(b) halogenating —O(CH₂)ₘOH substituent of the benzene to produce the benzene with —O(CH₂)ₘX substituent in the presence of an aprotic solvent, water, and halogenating agent at a temperature range of about 0° C. to about 90° C.; and
(c) cyclizing the compound produced in step (b) in the presence of alkyl lithium or aryl lithium to give ArX.

7. The process of claim 6, wherein the ArX is 6-bromo-2,3-dihydrobenzofuran.

8. The process of claim 7, wherein the temperature range in step (1) is about −40° C. to about −50° C.

9. The process of claim 8, wherein the phosphoramide reagent is N,N,N,N-tetra(C₁-C₆)-alkylphosphorodiamidic halide or N,N,N,N-tetraarylphosphorodiamidic halide.

10. The process of claim 9, wherein the phosphoramide reagent is N,N,N,N-tetramethylphosphorodiamidic chloride, N,N,N,N-tetramethylphosphorodiamidic bromide, N,N,N,N-tetraethylphosphorodiamidic chloride, N,N,N,N-tetraethylphosphorodiamidic bromide, N,N,N,N-tetraisopropylphosphorodiamidic chloride, N,N,N,N-tetraisopropylphosphorodiamidic bromide, N,N,N,N-tetraphenylphosphorodiamidic chloride, or N,N,N,N-tetraphenylphosphorodiamidic bromide.

11. The process of claim 10, wherein the base is selected from the group consisting of n-butyl lithium, phenyl lithium, potassium tert-butoxide, sodium hydride, lithium diusopropylamide, lithium diethylamide, lithium dimethylamide, potassium hexamethyldisilazide, sodium hexamethyldisilazide, and lithium hexamethyldisilazide.

12. The process of claim 11, wherein the base is sodium hexamethyldisilazide which is present in amounts between about 1 equivalents and about 6 equivalents relative to the amount of the phosphoramide reagent.

13. The process of claim 12, wherein the second aprotic solvent is TBF or a mixture of TEF and toluene.

14. The process of claim 13, wherein the temperature range in step (2) is about −20° C. to about 25° C.

15. A process for preparing a compound of Formula Ia: 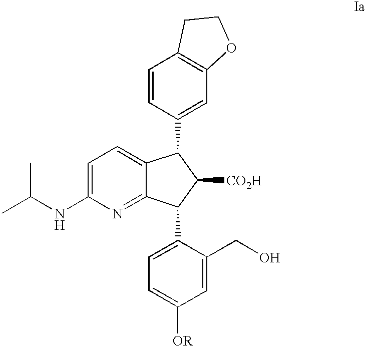
wherein R is independently H or (C₁-C₆)-alkyl comprising the steps of:
(1) reacting ArMgX reagent with a conjugate adduct of Formula IIa, 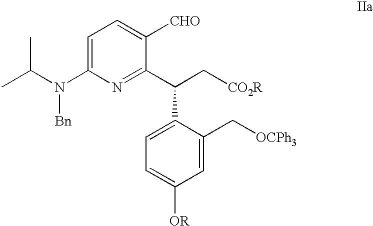
in the presence of a first aprotic solvent at a temperature range of about −80° C. to about 30° C. to give a Grignard addition product of Formula IIIa, and 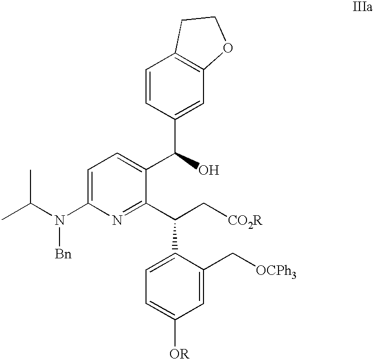
(2) adding phosphoramide reagent to a mixture of the Grignard addition product of Formula IIIa in a second aprotic solvent and a base at a temperature range of about −80° C. to about 30° C. to produce the desired compound of Formula Ia.

16. The process of claim 15, wherein the first or second aprotic solvent is selected from the group consisting of tetrahydrofuran, acetonitrile, dimethylacetamide, dimethylformamide, diethyl ether, N-methylpyrrolidinone, dichloromethane, methyl t-butyl ether, toluene, benzene, hexane, pentane, dioxane, and a mixture thereof.

17. The process of claim 16, wherein the first aprotic solvent is a 1:1 mixture of N-methylpyrrolidinone and tetrahydrofuran at a temperature range of about −40° C. to about −50° C. or N-methylpyrrolidinone at temperature range of about −20° C. to about −10° C.

18. The process of claim 17, wherein the Grignard reagent is

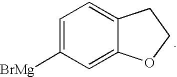

19. The process of claim 18, wherein the temperature range in step (1) is about −50° C. to about −40° C.

20. The process of claim 19, wherein the phosphoramide reagent is N,N,N,Netramethylphosphorodiamidic chloride, N,N,N,N-tetramethylphosphorodiamidic bromide, N,N,N,N-tetraethylphosphorodiamidic chloride, N,N,N,N-tetraethylphosphorodiamidic bromide, N,N,N,N-tetraisopropylphosphorodiamidic chloride, N,N,N,N-tetraisopropylphosphorodiamidic bromide, N,N,N,N-tetraphenylphosphorodiamidic chloride, or N,N,N,N-tetraphenylphosphorodiamidic bromide.

21. The process of claim 20, wherein the base is selected from the group consisting of n-butyl lithium, phenyl lithium, potassium tert-butoxide, sodium hydride, lithium diisopropylamide, lithium diethylamide, lithium dimethylamide, potassium hexamethyldisilazide, sodium hexamethyldisilazide, and lithium hexamethyldisilazide.

22. The process of claim 21, wherein the base is sodium hexamethyldisilazide which is present in amounts between about 1 equivalent and about 6 equivalents relative to the amount of the phosphoramide reagent.

23. The process of claim 22, wherein the second aprotic solvent is THF or a mixture of THF and toluene.

24. The process of claim 23, wherein the temperature range in step (2) is about −20° C. to about 25° C.

25. A process for preparing a compound of Formula Ia, 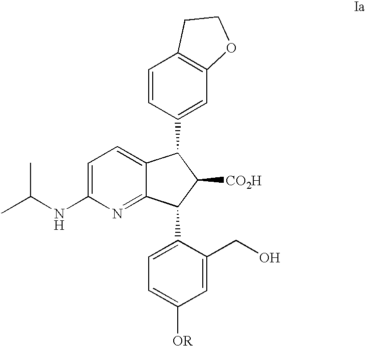
wherein R is independently H or (C₁-C₆)-alkyl comprising the steps of:
(1) reacting an α,β-unsaturated ester 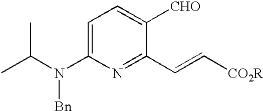
with a chiral auxiliary (S,S)-pseudoephedrine followed by treatment with an aryllithium compound 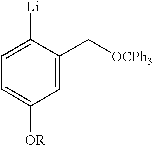
in toluene or tetrahydrofuran or a mixture thereof at a temperature range of about −80° C. to about 0° C. to give a conjugate adduct of Formula IIa, 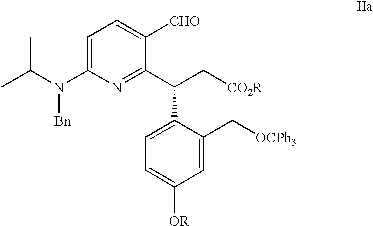
(2) reacting the conjugate adduct of Formula IIa with 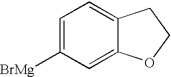
at a temperature range of about −80° C. to about 30° C. to give a Grignard addition product of Formula IIIa, 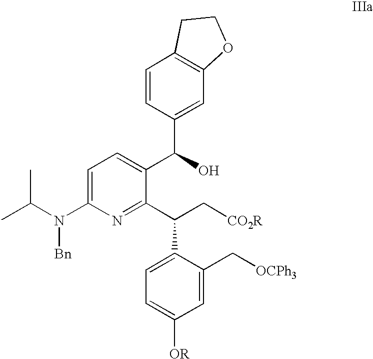
(3) adding phosphoramide reagent to a mixture of the Grignard addition product of Formula IIIa in the presence of tetrahydorfuran or a mixture of tetrahydrofuran and toluene, and a base at a temperature range of about −80° C. to about 30° C. to produce a cyclized compound of Formula IV, and 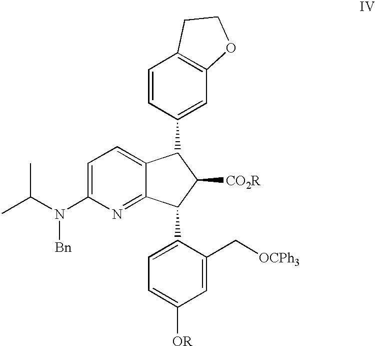
(4) removing protecting groups on the cyclized compound of Formula IV to give the desired compound of Formula Ia.

26. The process of claim 25, wherein the phosphoramide reagent is N,N,N,N-tetramethylphosphorodiamidic chloride, N,N,N,N-tetramethylphosphorodiamidic bromide, N,N,N,N-tetraethylphosphorodiamidic chloride, N,N,N,N-tetraethylphosphorodiamidic bromide, N,N,N,N-tetraisopropylphosphorodiamidic chloride, N,N,N,N-tetraisopropylphosphorodiamidic bromide, N,N,N,N-tetraphenylphosphorodiamidic chloride, or N,N,N,N-tetraphenylphosphorodiamidic bromide.

27. The process of claim 26, wherein the base is sodium hexamethyldisilazide which is present in amounts between about 1 equivalent and about 6 equivalents relative to the amount of the phosphoramide reagent.

28. The process of claim 27, which further comprises the steps of:
(a) deprotecting the cyclized compound of Formula IV by removing protecting groups with acid at a temperature range of about 0° C. to about 25° C.;
(b) crystallizing the deprotected compound as benzylamine salt; and
(c) hydrogenating the deprotected compound in the presence of a hydrogenation catalyst and a protic solvent at a temperature range of about 25° C. to about 40° C.

29. The process of claim 28, wherein the hydrogenation catalyst is Pd/C.

30. The process of claim 29, wherein the protic solvent is selected from the group consisting of (C₁-C₆) alcohol, H₂O and a mixture thereof.
